(19) United States
(12) Patent Application Publication
(10) Pub. No.: US 2014/0100102 A1
(43) Pub. Date: Apr. 10, 2014

(54) DEVICES AND METHODS FOR CELL LYSIS AND SAMPLE PREPARATION THROUGH CENTRIFUGATION

(71) Applicant: California Institute Of Technology, Pasadena, CA (US)

(72) Inventors: Aditya RAJAGOPAL, IRVINE, CA (US); Emil P. KARTALOV, LOS ANGELES, CA (US); Carl J. ALLENDORPH, LOS ANGELES, CA (US); Axel SCHERER, BARNARD, VT (US); Samson CHEN, FLUSHING, NY (US)

(73) Assignee: California Institute Of Technology, Pasadena, CA (US)

(21) Appl. No.: 14/048,735

(22) Filed: Oct. 8, 2013

Related U.S. Application Data
(60) Provisional application No. 61/711,842, filed on Oct. 10, 2012.

(51) Int. Cl. C12M 1/00

(52) U.S. Cl. CPC C12M 47/06
USPC 494/16; 494/50; 494/36; 494/37

(57) ABSTRACT

Methods and devices for molecular analysis are disclosed, based on centrifugation. A centrifuge device comprises strips of centrifuge tubes and elements to create a magnetic field. The magnetic shear forces applied to beads inside a solution with biological molecules permit the performance of different analytic techniques, such as lysis and sample preparation for PCR.

### CROSS REFERENCE TO RELATED APPLICATIONS

The present application claims priority to U.S. Provisional Patent Application No. 61/711,842, filed on Oct. 10, 2012, the disclosure of which is incorporated herein by reference in its entirety.

### TECHNICAL FIELD

The present disclosure relates to biomolecular analysis. More particularly, it relates to devices and methods for cell lysis and sample preparation through centrifugation.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIG. 1 depicts a top view of an exemplary sample preparation centrifuge.

FIG. 2 illustrates a cross-sectional view of an exemplary sample preparation centrifuge.

FIG. 3 illustrates an eight-tube strip with different receptacles.

FIG. 4 illustrates an exemplary cell lysis and sample preparation procedure.

FIG. 5 illustrates a top view of an exemplary tube arrangement.

FIG. 6 illustrates a top view of another exemplary tube arrangement.

FIG. 7 illustrates an exemplary centrifuge device.

FIG. 8 illustrates an exemplary electromagnet control diagram.

### SUMMARY

In a first aspect of the disclosure, a centrifuge device is described, the centrifuge device comprising: a rotating head; at least one centrifuge tube; at least one slot in the rotating head, configured to accept the at least one centrifuge tube; and at least one field element in the rotating head, wherein the at least one field element is configured to generate a magnetic field.

### DETAILED DESCRIPTION

The polymerase chain reaction (PCR) is a critical technique in the detection and amplification of nucleic acid products. However, before PCR can be performed, DNA must be liberated and purified from serological samples. While there are chemical kits that can be used to perform both lysis and nucleic acid purification, such methods require significant time-intensive, and highly-skilled technical labor to implement. The present disclosure describes several automated instruments and procedures that perform these tasks in a way that results in significant cost- and time-savings. As known to the person skilled in the art, lysis comprises breaking down the cell walls or membranes, thereby causing the liberation of intracellular molecules.

Specifically, devices and methods based on centrifugation are disclosed. In several embodiments of the disclosure, a device comprises a centrifuge suited for 100 uL-1 mL centrifuge tubes.

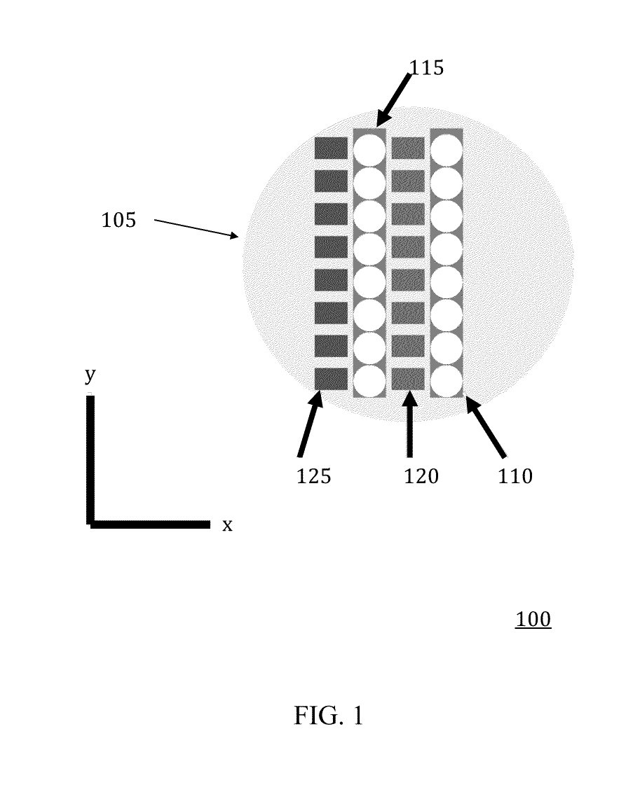

Referring to FIG. 1, in some embodiments the centrifuge device itself (100) comprises a rotating head (105) that rotates in a plane, which can be termed x-y plane. Centrifuge tubes are placed within through-holes or slots (110, 115) in the rotating head (105). In the example of FIG. 1, the through-holes (110, 115) amount to two rows of eight slots each for centrifuge tubes. By adjusting the rotational velocity and acceleration of the rotating head (105), fluids inside centrifuge tubes are accelerated centripetally in a controlled manner.

Furthermore, elements (120, 125) are present in the rotating head (105) which can generate a magnetic field. Elements (120, 125) are mounted normal to the x-y plane of rotation, thereby generating magnetic fields perpendicular to the axial dimension of the centrifuge tubes, as understood by the person skilled in the art. For example, elements (120, 125) can comprise electromagnets, activated by electric currents, or permanent magnets. Elements (120, 125) can be used to selectively accelerate particles inside centrifuge tubes in the slots (110, 115). As understood by the person skilled in the art, this acceleration is a function of the intensity of the magnetic field, the time-variance of the magnetic field, and the magnetic susceptibility of the accelerated particles.

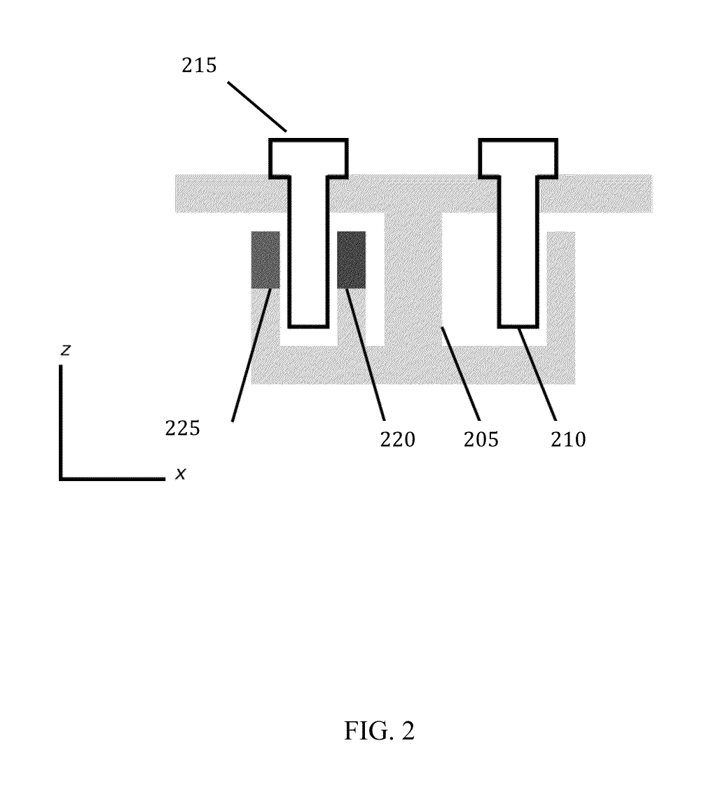

Referring to FIG. 2, a cross-sectional view of the structure of FIG. 1 is shown. In FIG. 2, the centrifuge superstructure (205) comprises the rotating head, and also comprises slots for a centrifuge tubes strip, for example a first eight-tube strip (210), and a second eight-tube strip (215). The second eight-tube strip (215) is situated between elements (220, 225) which can generate an electromagnetic field.

In several embodiments of the present disclosure, the centrifuge device is utilized with centrifuge tubes that are filled with both magnetic particles and non-magnetic, functionalized particles. As known in the art, functionalized particles are configured to attach to desired molecular species. The magnetic particles can be made from a variety of material including, but not limited to, iron, nickel, NeFeB, alinico, and cobalt. The non-magnetic particles can be fabricated from materials such as, but not limited to, polystyrene, silicon dioxide, quartx, aluminum oxide, and silicon.

Cellular samples are lysed by shear forces generated between accelerated magnetic particles and non-magnetic particles inside the tubes. Furthermore, chemically and biologically functionalized beads can be used for either selective or blind capture of nucleic acid targets. As understood by the person skilled in the art, selective capture is specific to certain molecular species, while blind capture is not selective. A filter can be included at the end of the tubes, the filter being sized to capture the functionalized beads but to allow the detritus and unwanted products to pass unimpeded. Detritus and unwanted products are flushed into a waste receptacle by centrifugation. Similar centrifugation is used to elute nucleic acid products into a secondary container.

In some embodiments, a type of receptacle may be used, for example a waste receptacle. In other embodiments, different receptacles may be used, or a combination of several receptacles. In some embodiments a tube may have more than one receptacle.

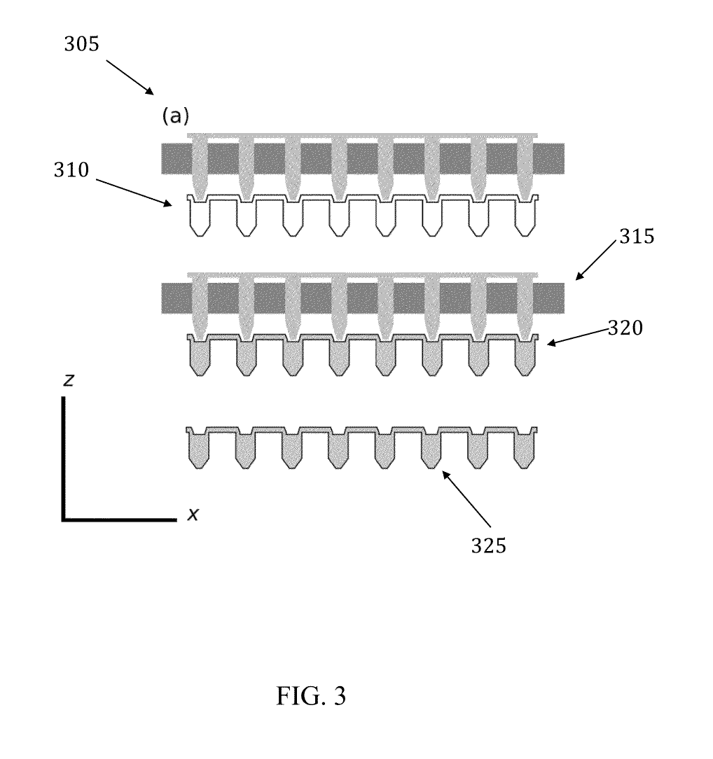

For example, FIG. 3 illustrates an eight-tube strip (305) with waste receptacles (310). Alternatively, an eight-tube strip (315) may have analysis receptacles (320) for polymerase chain reaction (PCR), or different biomolecular analysis techniques. An example of receptacles (325) separated from the tubes is also illustrated in FIG. 3.

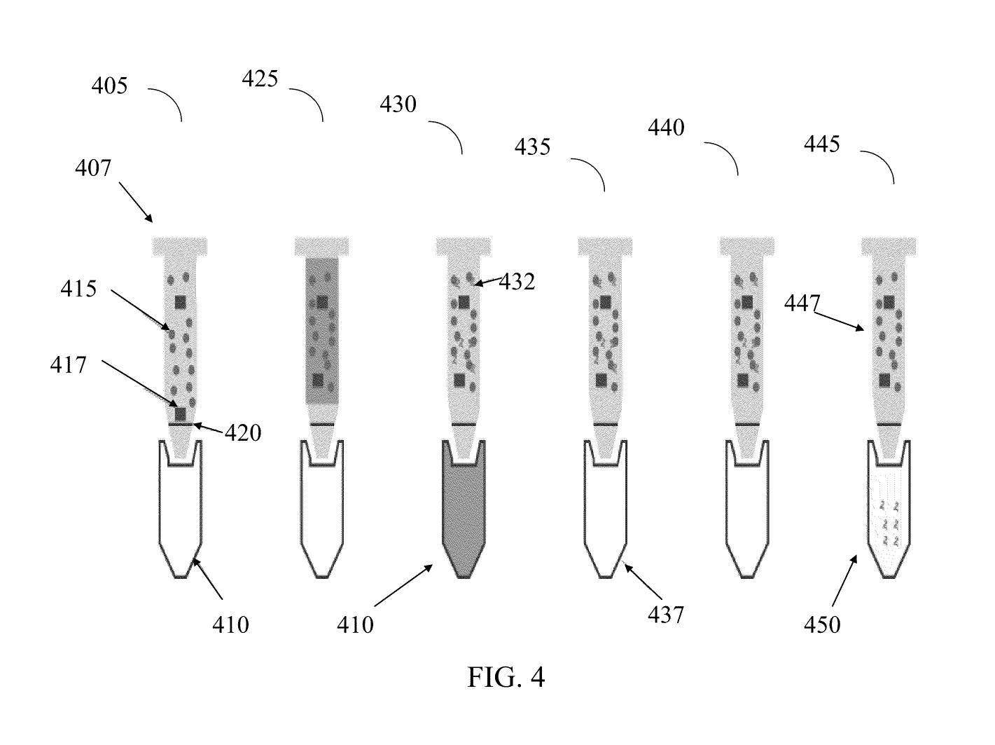

Referring now to FIG. 4, an exemplary cell lysis and sample preparation procedure is shown, comprising several steps.

In step (405), a sample preparation tube (407) is mated with a waste receptacle (410). The tube (407) may contain functionalized beads (415) and magnetic particles (417). The tube (407) can also comprise a bead capture filter (420).

In step (425), a sample analyte is added to tube (407) and a lysis protocol can be run. A lysis protocol as understood by the person skilled in the art may be used. For example, a lysis protocol is described in US Publication No. 2012/0175441 A1, published on Jul. 12, 2012, the disclosure of which is incorporated herein by reference in its entirety. Cellular lysis and nucleic acid binding can take place during the protocol. As part of the method, time may have to be allowed for newly liberated nucleic acid to bind to functionalized beads via diffusion.

Liberation of the nucleic acids occurs due to the shear forces between magnetic beads and functionalized beads, due to the movement of the centrifuge tubes within a magnetic field. The shear forces break down cellular walls, thereby liberating intracellular molecules, such as nucleic acids or other molecules of interest.

In step (430), waste is centrifuged into receptacle (410). The waste receptacle (410) can now be discarded. Liberated nucleic acid (432) are now present inside the tube.

In step (435), an analysis receptacle may be attached, for example a PCR product receptacle (437).

In step (440), elution buffer can be added, and an elution protocol can be run, as understood by the person skilled in the art.

In step (445), nucleic acid products are centrifuged into a tube (447) compatible with polymerase chain reaction. A DNA product capture receptacle (450) can be attached at the bottom of tube (447).

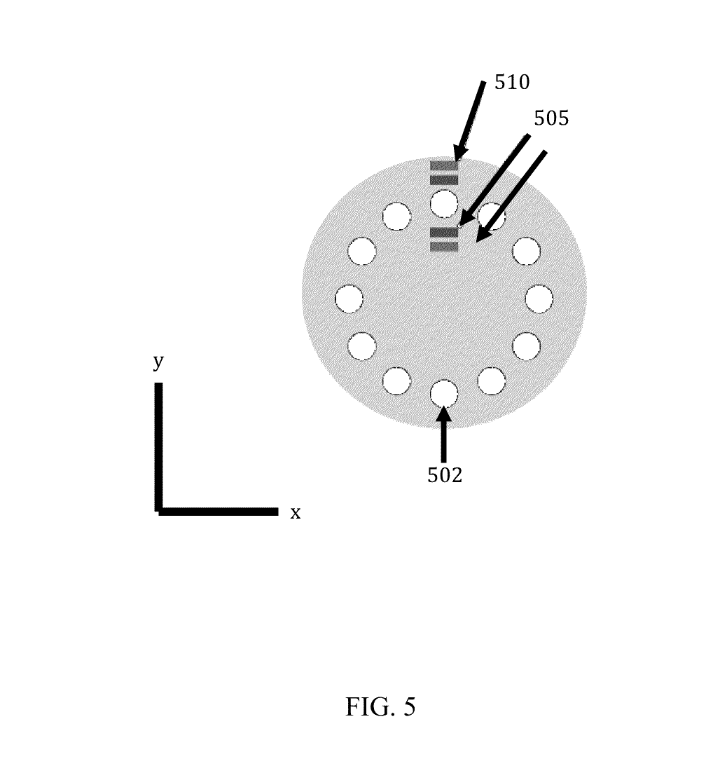

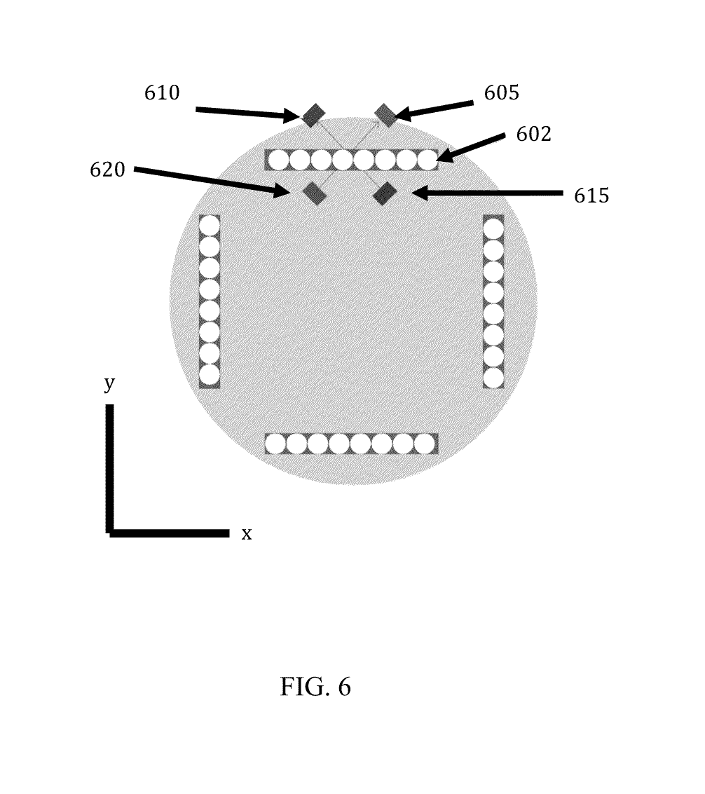

The platform geometry described in the present disclosure can be adjusted to accommodate either fewer or more centrifuge tubes. FIGS. 5 and 6 illustrate two examples of possible tube accommodations, from a top view. In FIG. 5, the tubes (502) are arranged in a circular fashion, and elements (505) and (510) can generate a magnetic field. In FIG. 6, a top view of a radial configuration of a 32-tube centrifuge platform is illustrated. Tubes (602) are arranged in four strips of eight tubes each. Elements (605), (610), (620) and (625) can generate a magnetic field. In one embodiment, a magnetic field is directed from element (615) to (610), and from element (620) to (605).

In several embodiments, a single set of electromagnets is used to agitate magnetic particles to shear cellular samples. As the centrifuge rotates, tubes are serially bought into the magnetic area of effect. While a tube is in this area, magnetic particles are agitated for cell lysis. By actuating two sets of electromagnets with orthogonal fields (for example as in FIG. 6), magnetic particles are selectively accelerated in three dimensions.

The methods described in the present disclosure are not limited to nucleic acid capture and PCR preparation. A similar process can be applied to protein sample preparation, for example, as understood by the person skilled in the art. Such process may also be applied to other types of sample preparation. For example, using immunoaffinity chromatography with ELISA chemistries, proteins can be captured in and separated from a cell analyte.

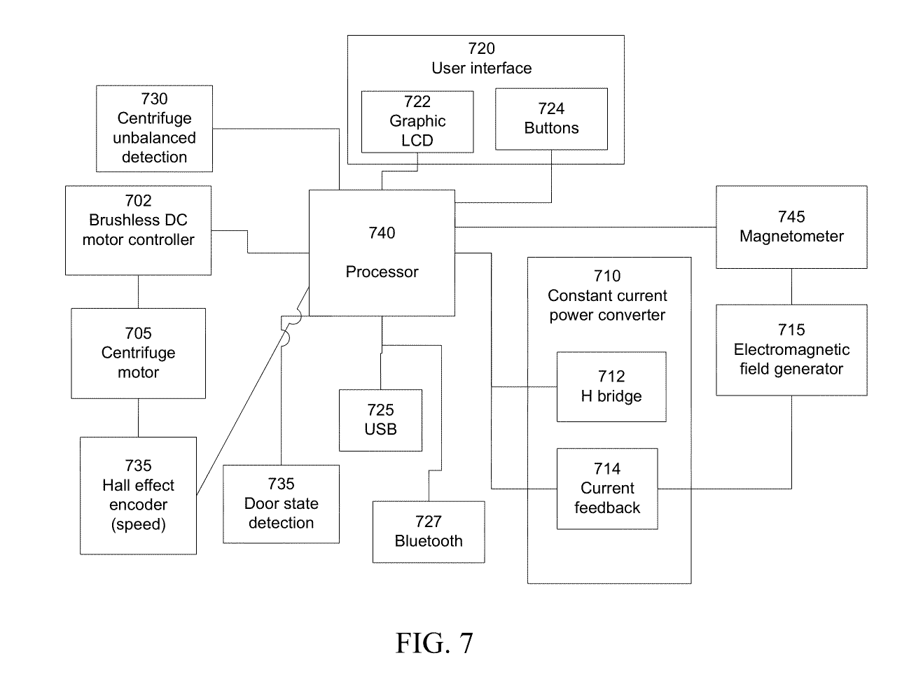

As illustrated in FIG. 7, in several embodiments of the disclosure, the main components of a centrifuge device comprise a Brushless DC (BLDC) motor controller (702) for spinning the centrifuge by controlling the centrifuge motor (705), and one or more constant current power controllers, or converters, (710), for driving a magnetic field generator (715). In addition, this device has a user interface (720) to allow a user to monitor the device's status, as well as to allow the user to control what type of cycle to run.

Controller (710) may comprise an H bridge (712) and a current feedback element (714). As understood by the person skilled in the art, an H bridge is an electronic circuit that enables a voltage to be applied across a load in either direction. User interface (720) may comprise a display such as an LCD (722) and control input mechanisms, such as buttons (724).

Communication between the centrifuge device and a host computer may be implemented by USB (725), Bluetooth (727), or other communication protocols.

The communication interface (725, 727) relays information about the state of the device, and also may report information about the magnetic fields, speeds, and any error conditions encountered during a run.

The centrifuge device may a variety of safety features to allow it to operate without damaging itself or the operator. For example, the centrifuge device may have a method of detecting if the centrifuge is unbalanced (730). Unbalanced centrifuges can shake violently, precess, and even ultimately cause injury or death. Circuit (730) can prevent the centrifuge device from running, if an out-of-balance condition is encountered.

Additionally, the centrifuge device may be equipped with a sensor (735) for determining the state of the centrifuge's lid. The centrifuge will not run if the lid is not securely closed.

A Hall effect encoder (735) can be used to regulate the speed of the motor (705).

A processor (740) may be used to regulate and control the different elements of a centrifuge device. A magnetometer (745) may also be part of the centrifuge device, allowing a measurement on the magnetic field generated by element (715).

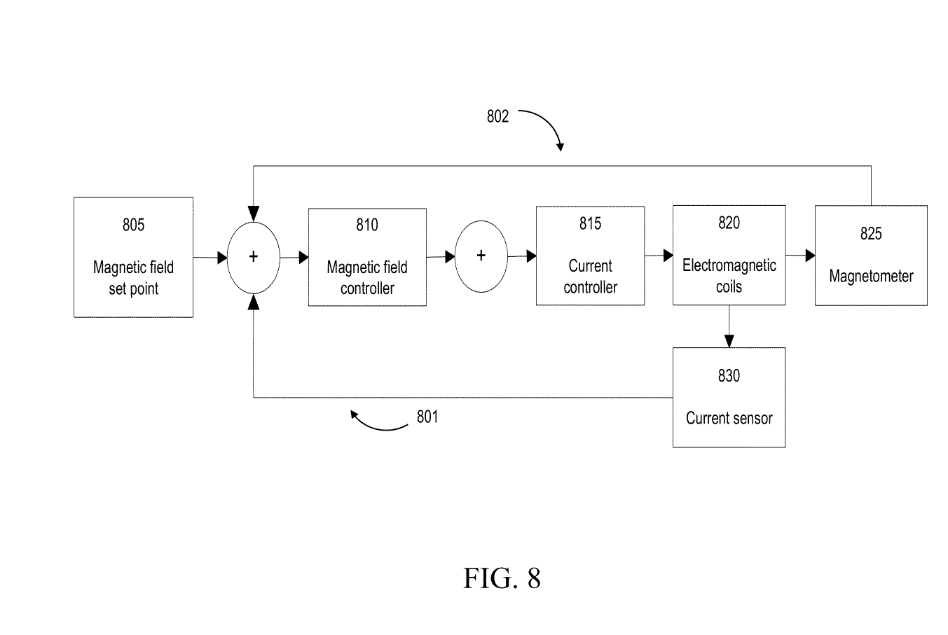

FIG. 8 illustrates an exemplary electromagnet controller block diagram. In several embodiments, the EM field control circuit uses two feedback control loops (801, 802) to maintain a constant magnetic field for driving the lysing elements.

The inner control loop (801) may comprise a constant current driving element that attempts to keep the current through the electromagnetic coils constant. Control loop (801) comprises a current controller (810), electromagnetic coils (820), a current sensor (830), and a magnetic field controller (810). The outer control loop (802) may use a magnetometer (825) to provide feedback of the actual magnetic field that is being exerted on the sample. The magnetic field set point (805) can then be controlled by a processor with a waveform intended to perform the lysis action.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art a complete disclosure and description of how to make and use the embodiments of the gamut mapping of the disclosure, and are not intended to limit the scope of what the inventor/inventors regard as their disclosure.

Modifications of the above-described modes for carrying out the methods and devices herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or devices, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

What is claimed is:

1. A centrifuge device comprising:
a rotating head;
at least one centrifuge tube;
at least one slot in the rotating head, configured to accept the at least one centrifuge tube; and
at least one field element in the rotating head, wherein the at least one field element is configured to generate a magnetic field.

2. The centrifuge device of claim 1, wherein the at least one centrifuge tube has a filter configured to allow disposal of waste products, while retaining desired products.

3. The centrifuge device of claim 2, wherein the desired products comprise chemically- and/or biologically-functionalized beads, and magnetic beads, wherein the functionalized beads are configured to attach to desired cells.

4. The centrifuge device of claim 3, wherein the functionalized beads and the magnetic beads are configured to selectively and/or blindly capture the desired cells.

5. The centrifuge device of claim 3, wherein the desired cells comprise nucleic acid within walls and/or membranes of the desired cells.

6. The centrifuge device of claim 5, wherein the desired cells are for lysis or polymerase chain reaction.

7. The centrifuge device of claim 2, further comprising at least one receptacle attached to a bottom end of the at least one centrifuge tube, wherein the at least one receptacle is for storing the waste products.

8. The centrifuge device of claim 1, further comprising at least one receptacle attached to a bottom end of the at least one centrifuge tube.

9. The centrifuge device of claim 8, wherein the at least one receptacle is for storing eluted nucleic acid products.

10. The centrifuge device of claim 1, wherein the at least one field element is a permanent magnet or an electromagnet.

11. The centrifuge device of claim 1, wherein the magnetic field direction is substantially perpendicular to an axial direction of the at least one centrifuge tube.

12. The centrifuge device of claim 1, further comprising at least one row of slots, the slots configured to accept centrifuge tubes.

13. The centrifuge device of claim 12, wherein the at least one row comprises eight slots.

14. The centrifuge device of claim 1, further comprising a radial arrangement of slots, the slots configured to accept centrifuge tubes.

15. The centrifuge device of claim 1, wherein the magnetic field comprises two perpendicular components, the two perpendicular components lying in a plane perpendicular to an axial direction of the at least one centrifuge tubes.

16. The centrifuge device of claim 1, wherein the at least one field element is fixed relative to the rotating head, and the at least one slot is configured to move through the magnetic field upon movement of the rotating head.

17. The centrifuge device of claim 3, wherein the functionalized beads are non magnetic.

18. A method comprising:
providing the centrifuge device of claim 1;
inserting a sample analyte in the at least one centrifuge tube, the sample analyte comprising cells, functionalized beads, and magnetic beads, wherein the functionalized beads are configured to attach to the cells; and
centrifuging the at least one centrifuge tube within the magnetic field, thereby causing shear forces between the cells attached to the functionalized beads and the magnetic beads, thereby lysing the cells, and thereby causing liberation of intracellular molecules.

19. The method of claim 18, further comprising disposing waste products into a first receptacle attached at a bottom end of the at least one centrifuge tube.

20. The method of claim 19, further comprising eluting the intracellular molecules into a second receptacle attached at a bottom end of the at least one centrifuge tube.

21. The method of claim 18, further comprising binding of the liberated intracellular molecules to the functionalized beads.

22. The method of claim 18, wherein the intracellular molecules are nucleic acids.
